(19) United States
(12) Patent Application Publication
(10) Pub. No.: US 2010/0098720 A1
(43) Pub. Date: Apr. 22, 2010

(54) Substance preventing adverse actions of therapeutic agents for dyslipidemia

(75) Inventors: Takashi Kadowaki, Tokyo (JP); Toshimasa Yamauchi, Tokyo (JP); Nobuo Mori, Gifu (JP); Osamu Sueno, Gifu (JP); Sachiko Kawamoto, Gifu (JP)

Correspondence Address: WENDEROTH, LIND & PONACK, L.L.P., 1030 15th Street, N.W.,, Suite 400 East, Washington, DC, 20005-1503, US

(73) Assignees: NIKKEN SOHONSHA CORPORATION; THE UNIVERSITY OF TOKYO

(21) Appl. No.: 12/385,961

(22) Filed: Apr. 24, 2009

(30) Foreign Application Priority Data
Oct. 20, 2008 (JP) 2008-269530

(51) Int. Cl. A61K 36/02

(52) U.S. Cl. 424/195.17

(57) ABSTRACT

A substance preventing adverse actions of conventional fibrate-series drugs as therapeutic agents for the metabolic syndrome and dyslipidemia to enhance the therapeutic effects thereof comprises an extract from Dunaliella salina or Dunaliella bardawil, for administration in combination with the fibrate-series drugs. The substance suppresses liver hypertrophy caused by the fibrate-series drugs as PPAR-α agonists and has an action of promoting fat combustion, suppressing fat synthesis and suppressing cell proliferation, to prevent disorders of liver functions.

### BACKGROUND OF THE INVENTION

1. Field of the Invention

The present invention relates to a substance preventing adverse actions of therapeutic agents for the metabolic syndrome and dyslipidemia, which contains Dunaliella as the active ingredient and is administered in combination with the therapeutic agents.

2. Prior Art

As such therapeutic agents, there have been known for example fibrate-series drugs such as “fenofibrate” (registered trademark) functioning for reducing neutral fat and low-density cholesterol possibly causing dyslipidemia and having an action as PPAR (peroxisome proliferation factor-activating receptor) α-agonist (receptor-activating agents).

Fibrate-series drugs (for example, fenofibrate) as the PPAR-α agonists regulate the expression of various proteins to improve the lipid metabolism, so that serum triglyceride and LDL cholesterol are reduced while HDL cholesterol is increased. The fibrate-series drugs with such effects are agents for lipid-improving so the agents have been used widely in clinical practice.

Herein, an agent for reducing fat cells using Dunaliella as well as foods and drinks therefor using Dunaliella have been known. The known agent for reducing fat cells contains yellowish orange algae of Dunaliella salina or Dunaliella bardawil of the genus Dunaliella of the order Vovocida of the class Chlorophyta or contains an extract obtained therefrom. Therefore, the agent is a highly safe, natural product with less adverse actions, functioning for reducing fat cells such as organ fat and reducing the amount of fat tissues. (JP-A-2007-210917).

However, the fibrate-series drugs (for example, fenofibrate) as PPAR-α agonists cause abnormalities in hepatic functions, as represented by for example AST, ALT and γ-GPT, and it is reported that the fibrate-series drugs have adverse actions such as choloplania, hepatopathy and the exacerbation of hepatopathy. According to reports about the results of clinical trials, abnormalities in numerical figures representing liver functions at laboratory tests are observed in 40% or more of patients on the administration of the fibrate-series drugs for 8 weeks.

The other report tells that the fibrate-series drugs cause liver hypertrophy in rodents, suggesting a possibility of liver cancer induction. Because no mechanism of liver hypertrophy has been elucidated yet, such fibrate-series drugs have been contra-indicated even for human patients with hepatopathy, although the adverse action more or less depends on species difference. Therefore, it is concerned that no conclusion would be made about some occurrence of adverse actions of the fibrate-series drugs in future.

It is understood that the agent for reducing fat cells as described in JP-A-2007-210917 has a safety profile with less adverse actions but the agent therefor is used as a pharmaceutical product to induce fat cells to disappear through apoptosis to reduce the number of fat cells. The patent reference never includes any reference to the suppression of adverse actions of other pharmaceutical products.

It is a problem to be solved by the invention to overcome various adverse actions of the fibrate-series drugs (for example, fenofibrate) as agents for lipid-improving, which are effective in the therapeutic treatment of the metabolic syndrome and dyslipidemia.

### SUMMARY OF THE INVENTION

As a specific approach for solving the problem, the invention provides a substance preventing adverse actions of therapeutic agents for dyslipidemia, the substance comprising an extract from Dunaliella salina or Dunaliella bardawil and having a potency to prevent adverse actions of fibrate-series drugs as the therapeutic agents for dyslipidemia, which is administered in combination with the fibrate-series drugs.

The substance preventing the adverse actions is prepared preferably in a form of capsules, tablets, granules or powders.

The substance preventing the adverse actions of the therapeutic agents for dyslipidemia according to the invention brings about an effect of suppressing liver hypertrophy caused by the fibrate-series drugs as PPAR-α agonists, when the substance comprises an extract from Dunaliella salina or Dunaliella bardawil. The suppression of liver hypertrophy is based on the promotion of fat combustion, the suppression of fat synthesis, and the suppressive action of cell proliferation. The substance exerts an excellent effect in preventing disorders of hepatic functions, together with the suppression of liver hypertrophy.

### BRIEF DESCRIPTION OF THE DRAWINGS

FIG. 1 is a graph showing the liver weight of KKA^{y} mice indicating the effect of the substance preventing such adverse actions and comprising the Dunaliella extract according to the invention as experimentally verified.

FIG. 2 is a graph showing the assay results of ACO involved in fat combustion as experimentally verified.

FIG. 3 is a graph showing the assay results of UCP2 involved in energy consumption as experimentally verified.

FIG. 4 is a graph showing the assay results of LPL involved in the decomposition of neutral fat as experimentally verified.

FIG. 5 is a graph showing the assay results of SCD1 involved in fat synthesis as experimentally verified.

FIG. 6 is a graph showing the assay results of TG content per whole liver weight as experimentally verified.

FIG. 7 is a graph showing the expression of the gene involved in the cell cycle as experimentally verified.

### DETAILED DESCRIPTION OF THE PREFERRED EMBODIMENTS

According to the invention, the extract from Dunaliella salina or Dunaliella bardawil suppresses more highly adverse actions occurring during the efficacious therapeutic treatment of dyslipidemia with the fibrate-series drugs, rather than the single use of the extract.

According to the invention, an extract from Dunaliella salina or Dunaliella bardawil is used. One example of the extraction approach comprises a first step of washing a dried powder of Dunaliella with ethanol, to which hexane is added under agitation, filtering the resulting mixture, and concentrating the filtrate, a second step of adding hexane to the resulting semisolid concentrate under agitation and filtering the resulting mixture to concentrate the filtrate, a third step of dissolving the concentrate in oily matter in hexane and leaving the resulting solution to stand alone to deposit solids, filtering and recovering the deposit, washing the deposit in ethanol and then drying the deposit to recover a powdery extract.

The Dunaliella extract thus obtained can be used as such powder as it is or may be formed into a formulation suitable for dosing, for example capsules prepared by filling the extract in desired capsule shapes, tablets, and granules.

The Dunaliella extract of the invention was experimentally examined using mice (KKA^{y}). The results are shown below.

### [Experiments]

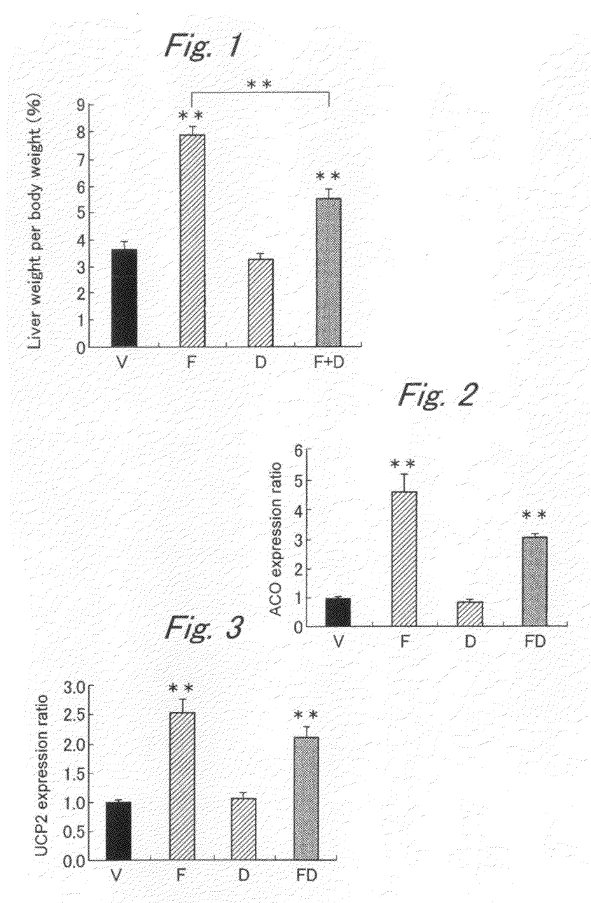

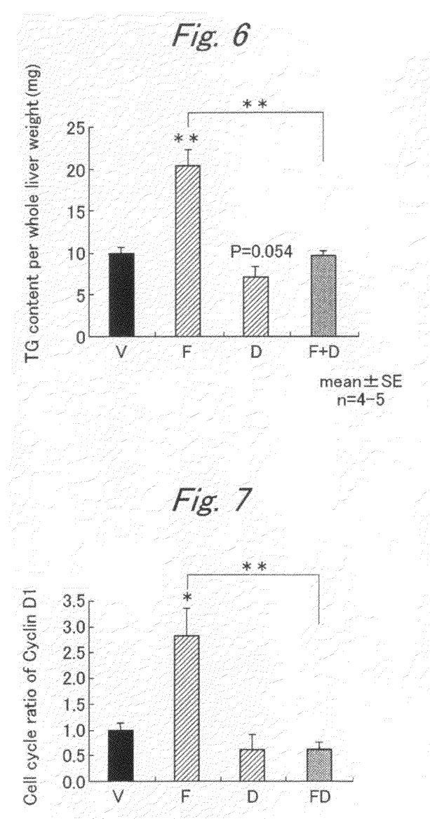

The KKA^{y} mice were fed with hyperfat diets and then grouped into group V (control group), group F (a group administered a fibrate-series drug alone), group D (a group administered the Dunaliella extract alone), and group FD (a group administered a combination of the fibrate-series drug and the Dunaliella extract). The fibrate-series drug and the Dunaliella extract were blended in feeds to 0.1% and 0.4%, respectively for dosing. Eight weeks after the administration, the mice were autopsied, to measure liver weight, ACO (acyl-CoA oxidase), UCP2 (Uncoupling protein 2), LPL (Lipoprotein lipase) and SCD1 (stearoyl-CoA desaturase-1). The results of the measurement are shown in FIGS. 1 to 5. Additionally, the TG (triglyceride) content in the whole liver weight and the cell cycle were also measured or counted. The results are shown in FIG. 6 and FIG. 7.

### [Evaluation]

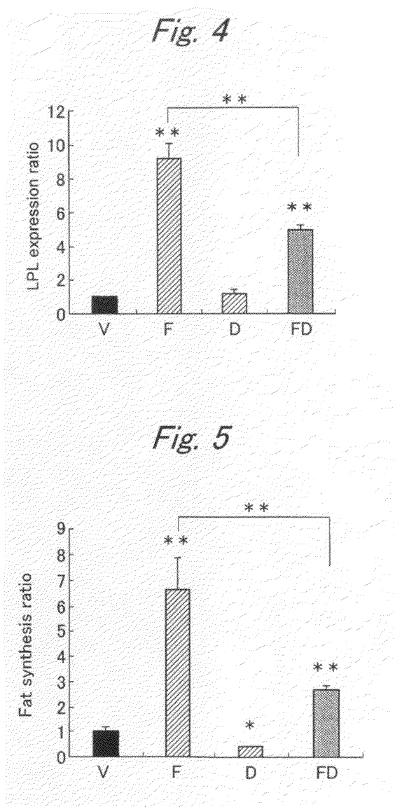

1) As apparent from FIG. 1, comparing the liver weight of the group FD with the group F, liver hypertrophy in the group FD was suppressed by about 30%.
2) As apparent from FIG. 2, the expression of ACO responsible for fat combustion is higher in the groups F and FD than in the group V, indicating the promotion of fat combustion in the groups.
3) As apparent from FIG. 3, the expression of the gene UCP2 involved in energy consumption was increased in the groups F and FD compared with the group V, indicating the occurrence of fat combustion in the groups.
4) As apparent from FIG. 4, the expression of LPL involved in neutral fat decomposition is increased in the groups F and FD more than in the group V, indicating the decomposition of neutral fat in the groups.
5) As apparent from FIG. 5, SCD1 involved in fat synthesis is suppressed by about 61.5% in the group FD compared with the group F, indicating the suppression of fat synthesis in the group FD. Compared with the group V, fat synthesis is suppressed in the group D, which possibly indicates the suppression of liver hypertrophy due to fat accumulation.
6) As apparent from FIG. 6, triglyceride (TG) per liver weight in the group FD is at a lower level by about 50% than in the group F, with no difference between the group FD and the group V, which may possibly suggest that triglyceride increase is suppressed in the group FD.
7) As apparent from FIG. 7, the gene responsible for cell cycle (cyclin D1) is abnormally high in the group F, showing the gene induces liver hypertrophy; in the group FD, however, the gene completely suppresses liver hypertrophy.

Based on the aforementioned results, it was found that a combined administration of the Dunaliella extract with the fibrate-series drug could suppress liver hypertrophy as the adverse action of the fibrate-series drug in the rodent. In the background, actions exist including the promotion of fatty acid combustion, the suppression of neutral fat synthesis and the suppression of cell proliferation. Together with the suppression of liver hypertrophy, disorders of liver functions caused by liver hypertrophy can be prevented.

This indicates that the extract can suppress adverse actions of PPAR-α agonists such as fibrate-series drugs (for example, fenofibrate) for use as therapeutic agents for dyslipidemia.

As described above, the Dunaliella extract of the invention is administered in combination with fibrate-series drugs for use as therapeutic agents for the metabolic syndrome and dyslipidemia, to suppress adverse actions of PPAR-α agonists such as fibrate-series drugs (for example, fenofibrate), such as liver hypertrophy to cause the disorders of liver functions. Via the synergistic effect of a combined use of both the Dunaliella extract and a fibrate-series drug, the effect of therapeutically treating the metabolic syndrome and dyslipidemia can significantly be enhanced, so the extract may highly possibly be used widely as a substance for use in combination of such drugs.

What is claimed is:

1. A substance preventing adverse actions of therapeutic agents for dyslipidemia, the substance comprising an extract from Dunaliella salina or Dunaliella bardawil for administration in combination with fibrate-series drugs as the therapeutic agents for dyslipidemia.

2. A substance preventing adverse actions of the therapeutic agents for dyslipidemia according to claim 1, wherein the extract is prepared in a form of capsules, tablets, granules or powders.
